(19) United States
(12) Patent Application Publication
(10) Pub. No.: US 2003/0150132 A1
(43) Pub. Date: Aug. 14, 2003

(54) Elastic sole pad

(76) Inventor: Su-Chu Lin, Taichung (TW)

Correspondence Address: Su-Chu Lin, 58, MA YUAN WEST ST., TAICHUNG, TW

(21) Appl. No.: 10/073,787

(22) Filed: Feb. 8, 2002

(51) Int. Cl. A61F 5/14

(52) U.S. Cl. 36/35 B; 36/153

(57) ABSTRACT

An elastic sole pad has an inlet tube, a plurality of vent holes, and a foamed polyurethane liquid injected into the elastic sole pad through the inlet tube. The elastic sole pad has a hollow interior to receive the foamed polyurethane liquid. The elastic sole pad is made of a soft and flexible plastics.

### BACKGROUND OF THE INVENTION

The present invention relates to an elastic sole pad. More particularly, the present invention relates to an elastic sole pad which has a foamed polyurethane liquid.

A foamed plastics outsole will be deformed and will lose elasticity after a long period of usage. Therefore, the foamed plastics outsole will become useless after a long period of usage.

### SUMMARY OF THE INVENTION

An object of the present invention is to provide an elastic sole pad which has a foamed polyurethane liquid therein so that the foamed polyurethane liquid will not leak while the elastic sole pad is pierced.

An object of the present invention is to provide an elastic sole pad which has a foamed polyurethane liquid therein so that the foamed polyurethane liquid will not lose elasticity after a long period of usage.

Accordingly, an elastic sole pad comprises an inlet tube, a plurality of vent holes, and a foamed polyurethane liquid. The elastic sole pad has a hollow interior to receive the foamed polyurethane liquid.

### BRIEF DESCRIPTION OF THE DRAWINGS

FIG. 1 is a perspective view of an elastic sole pad of a preferred embodiment in accordance with the present invention;

FIG. 2 is a perspective schematic view illustrating a pair of elastic sole pads are formed by a first mold and a second mold;

FIG. 3 is a schematic view illustrating a pair of elastic sole pads are disposed in a mold;

FIG. 4 is a schematic view illustrating a foamed polyurethane liquid is not injected into an elastic sole pad;

FIG. 5 is a schematic view illustrating a foamed polyurethane liquid is injected into an elastic sole pad; and

FIG. 6 is a schematic view illustrating an elastic sole pad is disposed in an outsole.

### DETAILED DESCRIPTION OF THE INVENTION

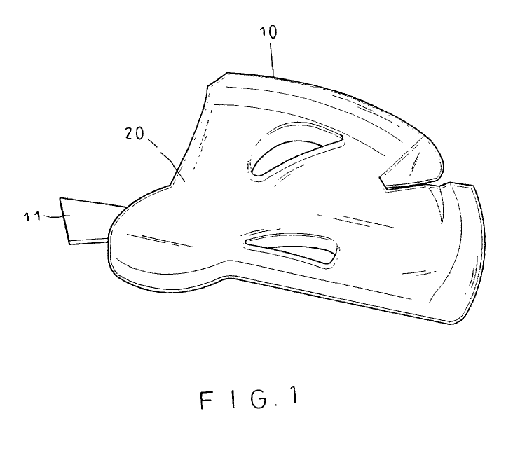

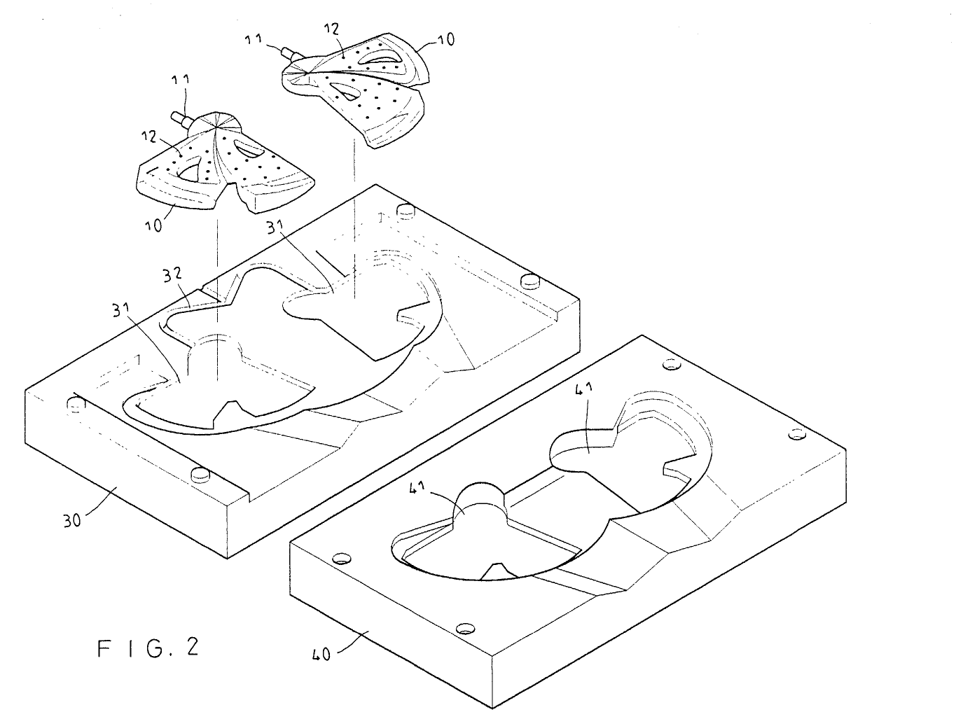

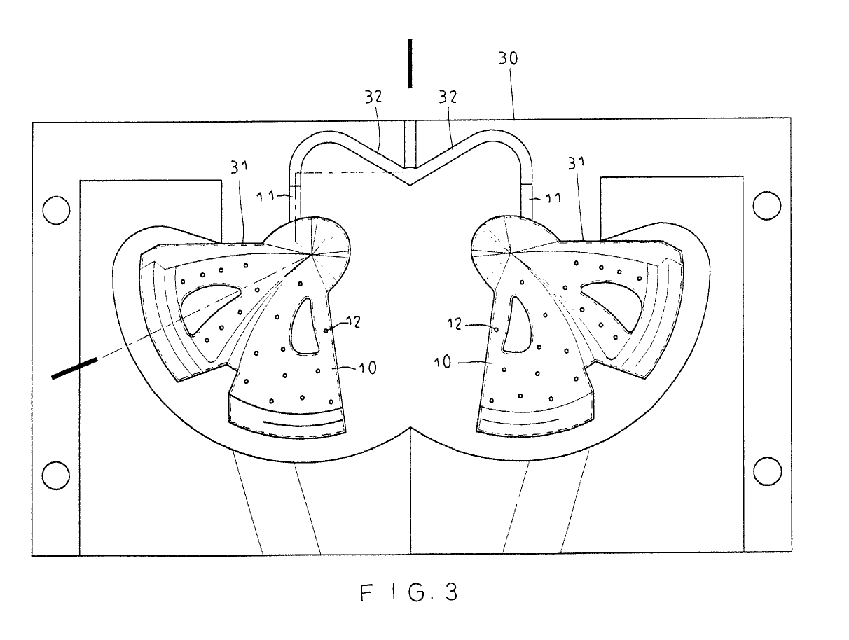

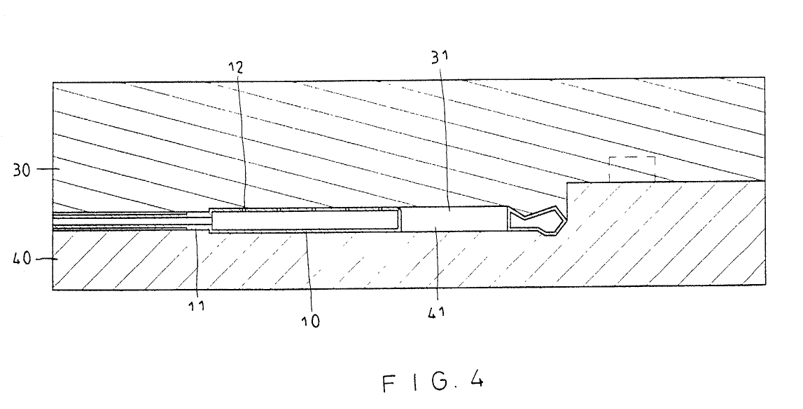

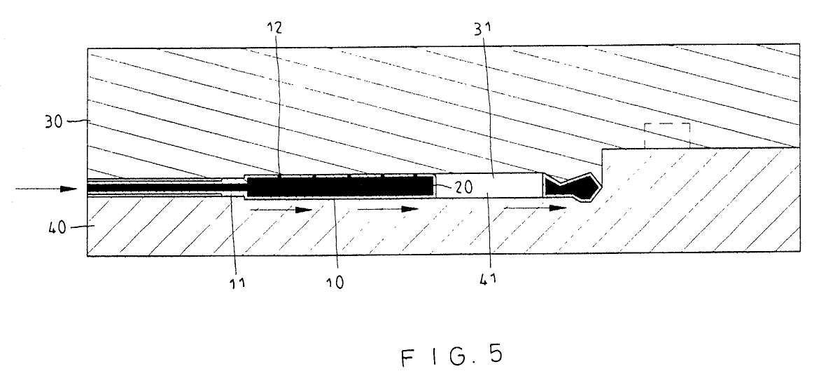

Referring to FIGS. 1 to 6, an elastic sole pad 10 comprises an inlet tube 11, a plurality of vent holes 12, and a foamed polyurethane liquid 20 injected into the elastic sole pad 10 through the inlet tube 11.

The elastic sole pad 10 has a hollow interior to receive the foamed polyurethane liquid 20 therein.

The elastic sole pad is made of a soft and flexible plastics.

The elastic sole pad 10 is disposed between a first mold 30 and a second mold 40.

The first mold 30 has a pair of first cavities 31 and a runner channel 32 communicating with the first cavities 31.

The second mold 40 has a pair of second cavities 41.

The first cavities 31 of the first mold 30 and the second cavities 41 of the second mold 40 receive a pair of elastic sole pad. Then the first mold 30 engages with the second mold 40 under a high pressure and a high temperature.

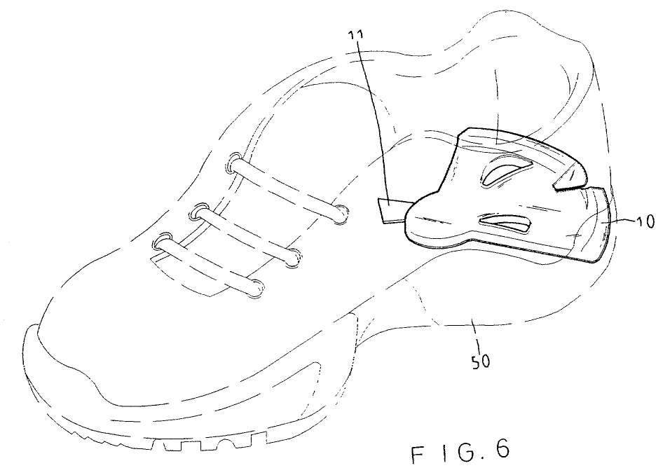

Referring to FIG. 6, the elastic sole pad 10 is disposed in an outsole 50.

The invention is not limited to the above embodiment but various modification thereof may be made. Further, various changes in form and detail may be made without departing from the scope of the invention.

I claim:

1. An elastic sole pad comprises:
an inlet tube,
a plurality of vent holes,
a foamed polyurethane liquid, and
the elastic sole pad having a hollow interior to receive the foamed polyurethane liquid therein.

2. The elastic sole pad as claimed in claim 1, wherein the elastic sole pad is made of a soft and flexible plastics.
